(19) United States
(12) Patent Application Publication
(10) Pub. No.: US 2023/0175931 A1
(43) Pub. Date: Jun. 8, 2023

(54) BACTERIA SAMPLING ROD

(71) Applicant: HO HONG JUNG AUTOMATION CO., LTD., New Taipei City (TW)

(72) Inventor: SHAY-CHIN SU, New Taipei City (TW)

(21) Appl. No.: 17/542,475

(22) Filed: Dec. 5, 2021

(51) Int. Cl. G01N 1/02

(52) U.S. Cl. CPC G01N 1/02; G01N 2001/028

(57) ABSTRACT

A bacteria sampling rod in the present disclosure comprises a rod body that has a comb teeth portion at the front end to contact with a culture dish through a user's operation similar to affixing a seal or making a rotation, catch and collect bacteria on the culture dish by the comb teeth portion, and transfer bacteria into a test vessel in which the bacteria solution is diluted for final analysis at a drug sensitivity test in an analyzer. The bacteria sampling rod can be installed on an automated machine properly for a full-automatic standardized operation as well as improved working performance and quality; the bacteria sampling rod with the advantage of an elastic buffering effect is free from the risk of a culture dish punctured during bacteria collection.

### BACKGROUND OF THE INVENTION

### 1. Field of the Invention

The present application relates to the field of biomedical inspection devices, particularly a bacteria sampling rod with respect to easy and safe bacteria collection and transfer for a full-automatic standardized operation of bacteria test and analysis.

### 2. Description of the Prior Art

In the field of biomedical inspection, bacteria to be tested are usually placed inside a culture dish for bacterial culture in a period of time. With bacterial culture completed, the bacteria in the culture dish are collected and transferred to a test vessel in which the bacteria solution is diluted and fed into an analyzer for analysis at a drug sensitivity test as well as collections of essential data.

In the prior art, the operation model for bacteria collection and transfer as mentioned previously is based on toothpicks or cotton buds with which bacteria in the culture dish are gathered by hand but criticized for low efficiency, a single sample size sustained uneasily, difficult maintenance of quality and realistic standardized work, and inconformity to modernized operation.

In addition, the technique for bacteria collection and transfer is unsatisfactory when a culture dish is punctured by force applied inappropriately that spoils bacteria collected and is accompanied with time and economy losses as well as some level of microbial contamination risks due to extra cleaning and disinfection measures for ambient environment exposed to bacteria probably.

Accordingly, the operation model for bacteria collection and transfer should be corrected; however, any attempt to change the biomedical inspection engineering, which refers to the highly complex and professional operation, will not be made by people who usually prefer not to take risk of failure. Thus, the work of bacteria collection disclosed hereinbefore has been done by hand basically for a long period of time and run counter to the modern engineering trend, i.e., automation. Accordingly, the issue for poor efficiency, safety and standardization of bacteria collection and transfer in traditional biomedical inspection engineering still deserves to be improved by persons skilled in the art.

### SUMMARY OF THE INVENTION

To overcome above drawbacks, the patent applicant provides a bacteria sampling rod comprising a comb teeth portion with which bacteria on a culture dish are caught effortlessly through a user's easy operation similar to affixing a seal or making a rotation for maintenance of a bacterial population and quality of the following test and analysis.

The patent applicant further provides a bacteria sampling rod which demonstrates an elastic buffering effect for no risk of a culture dish punctured as well as a safe and stable bacteria collection procedure.

The patent applicant further provides a bacteria sampling rod which can be installed on an automated machine properly for a full-automatic standardized operation procedure without excessive man-made interference as well as improved working performance and quality.

To this end, the bacteria sampling rod comprises a rod body that has a comb teeth portion at the front end to contact with a culture dish through a user's operation similar to affixing a seal or making a rotation, catch and collect bacteria on the culture dish by clearances in the comb teeth portion, and transfer bacteria into a test vessel in which the bacteria solution is diluted for final analysis at a drug sensitivity test in an analyzer.

In the above design, the comb teeth portion is made of soft materials through which bacteria are caught conveniently and a buffering effect is available in an operation of the rod body such as a vertical motion similar to affixing a seal or a rotational movement under application of a force for no puncture of a culture dish attributed to the comb teeth portion.

As mentioned in descriptions for a structural design hereinbefore, a bacteria sampling rod in the present disclosure can be installed on an automated machine properly for a full-automatic standardized operation procedure as well as improved working performance and quality; moreover, the bacteria sampling rod with the advantage of an elastic buffering effect is free from the risk of a culture dish punctured during bacteria collection for safety of a procedure to test and analyze bacteria.

Furthermore, the rod body is characteristic of a cavity designed inside.

Furthermore, the rod body features a tapering needle portion designed at the front segment and supplemented by reinforcing ribs peripherally.

Furthermore, the rod body comprises a notch on the top with which the rod body clutched by the automated machine is positioned but does not rotate idly.

### BRIEF DESCRIPTION OF THE DRAWINGS

FIG. 1 is a perspective view of a bacteria sampling rod;

FIG. 2 is a front view of a bacteria sampling rod;

FIG. 2a is an enlarged view for structure of a comb teeth portion in FIG. 2;

FIG. 3 is a schematic view for an operation of bacteria collected from a culture dish by a bacteria sampling rod; and

FIG. 4 is a schematic view for an operation of bacteria transferred to a test vessel by a bacteria sampling rod.

### DETAILED DESCRIPTION OF THE PREFERRED EMBODIMENT

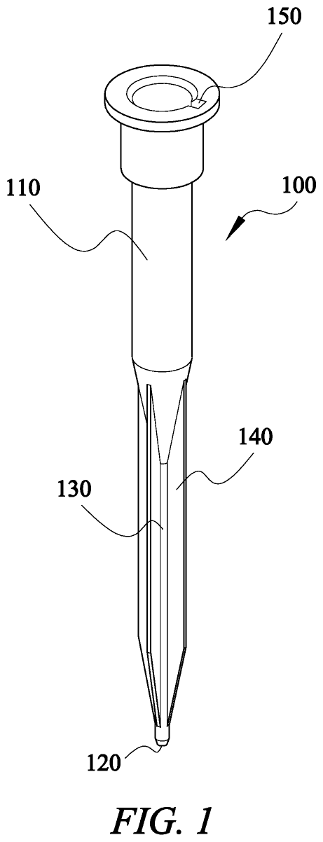

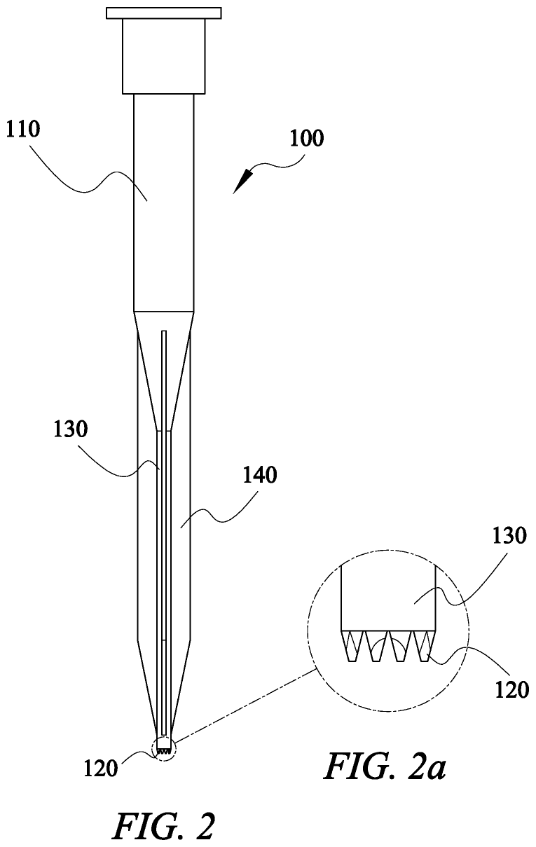

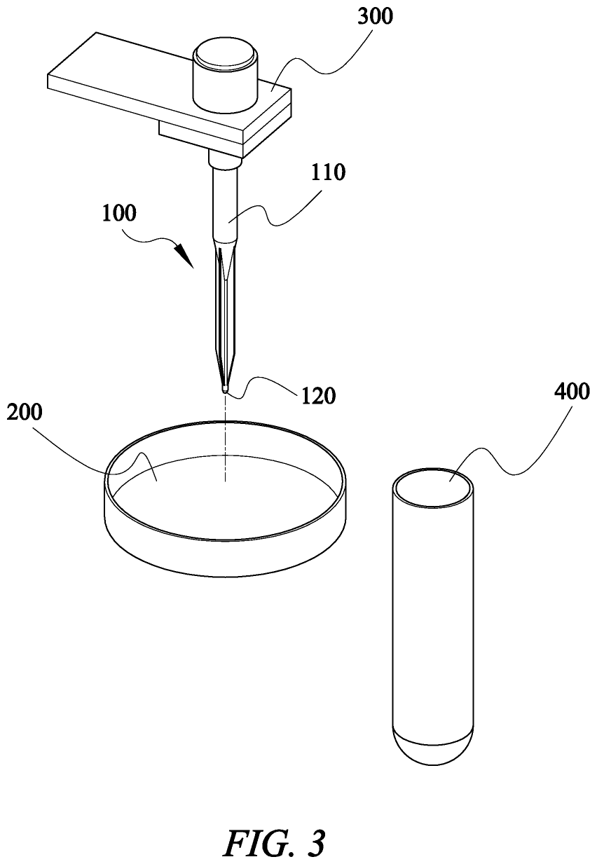

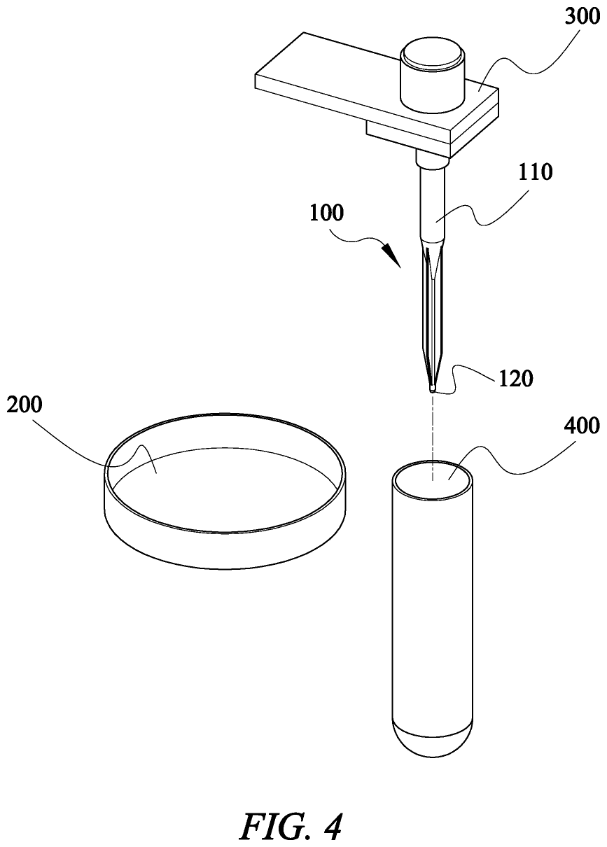

Referring to FIG. 1 to FIG. 4, which illustrate a bacteria sampling rod 100 in the present disclosure comprises a rod body 110 with a comb teeth portion 120 at the front end. The rod body 110 manipulated through a user's operation similar to affixing a seal or making a rotation contacts with a culture dish 200 from which bacteria are caught and collected smoothly through clearances at the comb teeth portion 120.

The rod body 110 is installed on an automated machine 300 for a full-automatic standardized operation procedure without excessive man-made interference as well as improved working performance and quality. First of all, the rod body 110 shifted by the automated machine 300 stays above the culture dish 200 and contacts with the culture dish 200 in a vertical motion similar to affixing a seal or a rotational movement during which bacteria in the culture dish 200 adhere to the comb teeth portion 120 smoothly and properly. Then, the rod body 110 is moved to a test vessel 400 by the automated machine 300 for completion of bacteria collection and transfer. Finally, the bacteria solution in the test vessel 400 is diluted and fed into an analyzer for inspection and analysis at a drug sensitivity test.

In the design of the comb teeth portion 120 of the bacteria sampling rod 100, the comb teeth portion 120 is made of soft materials or further manufactured as a soft laminate with a threadlike appearance through which the culture dish 200 is touched after application of a force for adhesion of bacteria easily. Moreover, because of a buffering effect from the soft comb teeth portion 120, an incident at the culture dish 200 punctured by the comb teeth portion 120 of the rod body 110 moving vertically or rotationally under application of a force is avoided effectively for a safe and stable sampling process. The rod body 110 further comprises a notch 150 on the top with which the rod body 110 clutched by the automated machine 300 is positioned but does not rotates idly.

Furthermore, the bacteria sampling rod 100 is a consumable with a cavity designed inside for saving materials and satisfying appropriate strength.

Alternatively, the rod body 110 comprises a tapering needle portion 130 designed at the front segment for accurate positioning and supplemented by reinforcing ribs 140 peripherally for structural stability.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A bacteria sampling rod, comprising:
a rod body, which has a comb teeth portion at the front end to contact with a culture dish in a user's operation similar to affixing a seal or making a rotation and collect bacteria on the culture dish by clearances in the comb teeth portion.

2. The bacteria sampling rod as claimed in claim 1 wherein the comb teeth portion is made of soft materials through which bacteria are caught conveniently and a buffering effect is available in an operation of the rod body such as a vertical motion similar to affixing a seal or a rotational movement under application of a force for no puncture of a culture dish attributed to the comb teeth portion.

3. The bacteria sampling rod as claimed in claim 1 wherein the rod body is characteristic of a cavity designed inside.

4. The bacteria sampling rod as claimed in claim 1 wherein the rod body features a tapering needle portion designed at the front segment and supplemented by reinforcing ribs peripherally.

5. The bacteria sampling rod as claimed in claim 1 wherein the rod body comprises a notch on the top with which the rod body clutched by the automated machine is positioned but does not rotate idly.
